(19) United States
(12) Patent Application Publication
(10) Pub. No.: US 2015/0328249 A1
(43) Pub. Date: Nov. 19, 2015

(54) MODULATION OF MYOFIBER REPAIR BY ANTI-MYOSTATIN STRATEGIES AND/OR PPAR GAMMA LIGANDS, ALONE OR IN COMBINATION WITH STEM CELLS, FOR THE THERAPY OF CRITICAL LIMB ISCHEMIA AND OTHER ISCHEMIC PROCESSES AFFECTING THE SKELETAL MUSCLE

(71) Applicant: LOS ANGELES BIOMEDICAL RESEARCH INSTITUTE AT HARBOR-UCLA MEDICAL CENTER, Torance, CA (US)

(72) Inventors: Nestor F. Gonzalez-Cadavid, Pasadena, CA (US); Rodney A. White, San Pedro, CA (US)

(73) Assignee: Los Angeles Biomedical Research Institute at Harbor-UCLA Medical Center, Torrance, CA (US)

(21) Appl. No.: 14/651,616

(22) PCT Filed: Dec. 11, 2013
(86) PCT No.: PCT/US2013/074467

§ 371 Date: Jun. 11, 2015

Related U.S. Application Data
(60) Provisional application No. 61/735,953, filed on Dec. 11, 2012.

(51) Int. Cl. A61K 31/7105; A61K 31/4439; A61K 45/06; A61K 38/39; C07K 16/22; C12N 15/113; A61K 35/545; A61K 38/17

(52) U.S. Cl. CPC A61K 31/7105; A61K 35/545; A61K 31/4439; A61K 38/1709; A61K 38/39; C07K 16/22; C12N 15/1136; A61K 45/06; C12N 2320/31; C12N 2310/11; C12N 2310/141

(57) ABSTRACT

Treating or inhibiting an ischemic condition affecting the skeletal muscle comprising administering an agent having a property to inhibit an activity or a protein expression of myostatin or growth differentiation factor 8 (GDF-8) according to a regimen to treat or inhibit the ischemic condition. Treating or inhibiting an ischemic condition affecting the skeletal muscle including administering an effective amount of a thiazolidenedione or other PPAR gamma agonist at a dosage that do not exert glycemic control or induce overweight. A kit for use in treating or inhibiting an ischemic condition affecting the skeletal muscle comprising a quantity of an agent having a property to inhibit an activity or a protein expression of myostatin or growth differentiation factor 8 (GDF-8) and/or a quantity of a thiazolidenedione or other PPAR gamma agonist and instructions for administration of a dosage of that quantity according to a long term continuous regimen.

### BACKGROUND

Critical limb ischemia (CLI) is a devastating disease that affects mainly patients with type 2 diabetes mellitus (T2DM) and obesity, with high risk of amputation of lower extremities and post-surgery mortality, and no effective medical treatment. Stem cell therapy is promising, and bone marrow mesenchymal stem cells and endometrial regenerative cells (ERC) are in clinical trials based on their angiogenic capacity, but their myogenic capacity for the repair of the necrotic skeletal muscle myofibers or their antifibrotic effects are unexplored.

T2DM, obesity, and metabolic syndrome have reached an epidemic proportion in the United States, affect disproportionately minorities, and their complications are more severe. One of the most serious cardiovascular complications of T2DM is peripheral artery disease (PAD), that afflicts over 8 million people. The most severe form of PAD is CLI, in which blood flow is insufficient to maintain tissue viability causing extreme chronic pain, non-healing ulcers, or gangrene in the leg/foot, due to neuropathy and necrosis of the skeletal muscle, arteries and other tissues. CLI has an incidence of 300,000 cases/year, is the leading cause of non-traumatic amputation, causing 120,000 amputations per year, and the relative risk of amputation is 40 times greater in the diabetic population. Approximately 20-45% of patients require amputation and the one year mortality may be close to 45% after amputation. The very poor quality of life may be comparable to that experienced by cancer patients.

CLI is characterized by a defective revascularization compensatory response, that normally ensues after ischemia injury, driving both angiogenesis (branching of a new small vessel from an existing vessel) and arteriogenesis (enlargement of arterioles maturing into conducting arteries). Angiogenic processes are triggered by an early inflammation stage with upregulation of hypoxia inducible factor (HIF) that stimulates the expression of vascular endothelial growth factor (VEGF), angiopoietins, and nitric oxide (NO), leading to cytokines and MMP release, vasodilation and endothelial cell migration. There is cellular remodeling and proliferation, and matrix degradation and synthesis. Arteriogenesis is initiated by fluid shear stress, and requires the recruitment of circulating endothelial progenitor cells and bone marrow derived mononuclear cells. The impact of the uncompensated and aggravated ischemia, particularly in T2DM, on the skeletal muscle and other tissues leads to their extensive necrosis.

Conventional treatments include angioplasty and/or bypass to remove blood vessel blockage, along with drugs for ulcer recovery and wound healing, and debridement of damaged/infected tissue, focusing on: (a) prevention of amputation, (b) facilitation of wound healing, (c) stimulation of angiogenesis and tissue repair. However, surgical or endovascular revascularization approaches and medical therapy stimulating angiogenesis are of limited or no efficacy, and amputation is then needed. Therefore, CLI is an excellent target for immediate translation to the clinic of novel vascular regeneration therapies for both angiogenesis and arteriogenesis, as well as for skeletal muscle repair, based on studies in mice, rat, and rabbit models, with ischemia in the hind limb legs, and specifically in diabetes in mice and rats.

Stem cells are promising for revascularization in PAD and limb ischemia in the animal models, mainly bone marrow mesenchymal and adipose tissue derived stem cells, and ERC, but few have been tested in true T2DM-CLI models, and none focusing on pharmacological or stem cell cross-talk modulation. Various degrees of improvement in blood flow, capillary density, angiogenic factors, muscle atrophy, interstitial fibrosis, skin ulcers, etc, have been reported. Several clinical trials are ongoing (mostly outside the United States) with autologous stem cells, and although no serious side effects have been observed, the angiogenic effects are limited, the repair of skeletal muscle necrosis is not defined, and the autologous stem cells isolation is too invasive for CLI patients.

### SUMMARY

A major hurdle of stem cells therapy for necrotic diseases of the skeletal muscle and other tissues, particularly in T2DM, and obviously in CLI, is the noxious terrain where these cells are implanted and that also impair the endogenous stem cells. To overcome this, a strategy is required that not only would modulate stem cells differentiation into the right lineages, but that would also act directly on the host tissue counteracting processes that oppose tissue repair. Three types of pharmacological modulators of stem cells implants may be used to stimulate their efficacy in skeletal muscle repair in CLI.

First, the therapeutic inhibition of myostatin expression, the key inhibitor of skeletal muscle mass, and a diabetogen, obesogen, profibrotic, anti-PPARγ, and anti-angiogenic factor. Myostatin knockout mice and cattle are considerably hypermuscular whereas myostatin overexpression transgenic mice are underweight or frankly caquexic. In other contexts it is known that myostatin expression and/or activity may be blocked or inhibited by the following types of agents: a) follistatin or decorin, to bind and inactivate myostatin; b) myostatin neutralizing antibodies to exert similar effects; c) myostatin antisense or siRNA to block the expression of myostatin mRNA or its translation into protein; d) myostatin propeptide to bind and inactivate myostatin; e) ligands of the myostatin receptor, the activin type IIb receptor (ActIIbR), to block myostatin signaling; f) microRNAs against or pro-myostatin to control the synthesis of mRNAs.

Second, administration of peroxisome-proliferator-activated receptor γ (PPARγ) agonists, since this is the nuclear receptor in T2DM, obesity, inflammation, and oxidative stress that is activated by endogenous ligands (free fatty acids) and possibly other obesogens, or by pharmacological ligands, e.g. pioglitazone and other thiazolidenediones (TZD) (FIG. 1). Pioglitazone is used clinically as ACTOS for insulin resistance and T2DM, and experimentally acts as antifibrotic, anti-inflammatory, anti-oxidative stress, and proangiogenic and also as a key modulator of stem cells proliferation and multiple differentiation. TZD angiogenic effects in the diabetic vasculature occur via HIF/VEGF, contrasting with their anti-angiogenesis in other settings.

Third, agents that modulate the nitric oxide (NO)/cGMP pathway, specifically molsidomine an NO generator tested in clinical trials, that stimulates blood flow, angiogenesis, and muscle repair in the skeletal muscle. In animal models, NO, mainly from endothelial nitric oxide synthase (eNOS) and through cGMP production, is essential for revascularization of the ischemic hind limb, by acting as vasodilator and inducing VEGF, fibroblast growth factor and urokinase-type plasminogen activator. eNOS overexpression, and the NOS substrate L-arginine stimulate angiogenesis. L-arginine in the diet increases endothelium vasodilation, and improves the clinical symptoms of intermittent claudication in PAD patients, but only sporadic studies or case reports are available in CLI with potent NO generators or PDE5i alone and none together with stem cells. NO and cGMP modulate stem cell differentiation.

Accordingly, a therapy of critical limb ischemia (CLI), peripheral arterial disease (PAD) and of other ischemic conditions affecting the skeletal muscle should:

1) target preferentially skeletal muscle repair and not just angiogenesis, by promoting myofiber repair and inhibiting lipofibrotic degeneration;

2) focus on counteracting or preventing the increase in the main inhibitor of skeletal muscle mass and profibrotic factor, myostatin or growth differentiation factor 8 (GDF-8), induced in the skeletal muscle by CLI and/or by the administration of stem cells, by utilizing anti-myostatin approaches, specifically: a) follistatin, b) decorin; c) myostatin antibodies; d) myostain propeptide; e) myostatin shRNA or antisense RNA; f) myostatin microRNAs inhibiting myostatin expression or anti-microRNAs stimulating this expression; g) ligands of the myostatin receptor, the activin type IIb receptor (ActIIbR)

3) this approach should also focus on a combination of the anti-myostatin strategy with the use of stem cells, specifically muscle derived stem cells (MDSC), but any other type as well, that thus will be allowed to proceed in their differentiation or paracrine effects to the late stage of myofiber formation and to the inhibition of fibrosis, by neutralizing the noxious effects triggered by the stem cells themselves;

4) the anti-myostatin approach, alone or in combination with stem cells, or the use of stem cells alone, may also be complemented with thiazolidenediones, specifically pioglitazone or other PPARγ agonists, given long-term continuously at low doses that do not exert anti-glycemic effects or cause overweight and fat accumulation, in order to combat skeletal muscle fibrosis, chronic inflammation and promote exogenous or endogenous stem cell differentiation for myofiber repair. Long-term continuous administration as described herein is months or years of administration according to a regimen schedule or period (e.g., daily, twice-daily, every other day, weekly, etc. depending on, for example, dosage).

### BRIEF DESCRIPTION OF THE DRAWINGS

FIG. 1 Systemic effects of ligand binding to PPARγ in diabetes and obesity. Solid arrow: stimulation; broken arrow: down regulation.

FIG. 2 MDSC alone, or supplemented with molsidomine, decreases mortality and prevent leg loss in CLI. The appearance of ischemic legs at 3 weeks after single femoral artery ligation (SFAL) (n=8/group) is shown. D: death.

FIG. 3. MDSC alone, or to a much lesser degree when supplemented with molsidomine, increase stem cell number in the skeletal muscle during CLI. The gastrocnemius from the ischemic legs from the mice on FIG. 2 were subjected to immuno-histochemistry for stem cell marker Oct 4. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with molsidomine alone. *; p<0.05; ***: p<0.001.

FIG. 4. Critical limb ischemia is associated with angiogenesis stimulation in the skeletal muscle during CLI that is further increased by MDSC alone and the other treatments. The muscle tissues were subjected to immunohistochemistry for VGEF. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with molsidomine alone. ***: p<0.001.

FIG. 5 Since MDSC alone increase calponin, a specific marker of smooth muscle cells, while decreasing the ASMA/calponin ratio, it may be assumed that vascular smooth muscle cells are increased in the skeletal muscle during CLI and hence angiogenesis, while myofibroblasts are decreased. The muscle tissues were subjected to immunohistochemistry for calponin. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with molsidomine alone. *; p<0.05; ***: p<0.001.

FIG. 6 The pro-angiogenesis stimulation by MDSC in the skeletal muscle during CLI alone leads to the overexpression of CD31, a marker of vascular endothelium in blood vessels The muscle tissues were subjected to immunohistochemistry for CD31. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with.

FIG. 7 The proangiogenesis stimulation by MDSC alone in the skeletal muscle during CLI is confirmed with another vascular endothelial marker The muscle tissues were subjected to immunohistochemistry for von Willebrandt factor. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with molsidomine alone. *; p<0.05.

FIG. 8 Critical limb ischemia is associated with fat infiltration in the skeletal muscle that is counteracted by MDSC alone or supplemented with molsidomine, but not with molsidomine alone. The muscle tissues were subjected to histochemistry with Oil red O. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with molsidomine alone. *; p<0.05; **: p<0.01; ***: p<0.001.

FIG. 9 MDSC alone, or in combination with molsidomine, exert a neurotrophic effect in the skeletal muscle during CLI by increasing BDNF. The muscle tissues were subjected to immunohistochemistry for BDNF. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with molsidomine alone. *; p<0.05; **: p<0.01.

FIG. 10 MDSC alone, or the other treatments but to a lesser degree, reduce apoptotic cell death in the skeletal muscle during CLI and hence protect myofibers. The muscle tissues were subjected to immuno-histochemistry for apoptosis by TUNEL. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with molsidomine alone. **: p<0.01; ***: p<0.001.

FIG. 11 MDSC alone, but not the other treatments, stimulate the early phase of myofiber repair in the central nuclei by increasing central nuclei during CLI. The muscle tissues were subjected to hematoxylin-eosin histochemistry. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with molsidomine alone. ***: p<0.001.

FIG. 12 Surprisingly, no treatment resulted in the stimulation of the early phase of myogenesis in the skeletal muscle during CLI. The muscle tissues were subjected to immunohistochemistry for the early myogenesis gene, MyoD. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with molsidomine alone. *; p<0.05.

FIG. 13 The lack of stimulation of early myogenesis in the skeletal muscle during CLI by MDSC was accompanied by even a decrease in the late phase of myofiber formation. The muscle tissues were subjected to immunohistochemistry for the late myogenic gene, MHC-II. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with molsidomine alone. *; p<0.05.

FIG. 14 The lack of effects on late myofiber formation by MDSC alone are accompanied by an increased collagen deposition in the skeletal muscle, indicating fibrosis. The muscle tissues were subjected to histochemistry with Masson trichrome. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with molsidomine alone. **: p<0.01; ***: p<0.001.

FIG. 15 The ineffective late myofiber repair and increased fibrosis in the skeletal muscle during CLI by MDSC alone may be related to the unexpected overexpression of the muscle mass inhibitor myostatin in the untreated CLI, a process that is even further stimulated by MDSC alone implantation. The muscle tissues were subjected to immunohistochemistry for myostatin. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with molsidomine alone. *; p<0.05; **: p<0.01; ***: p<0.001.

FIG. 16 The overexpression of myostatin in the skeletal muscle during CLI that is exacerbated by MDSC implantation is not balanced by an overexpression of follistatin, a protein that binds and inactivates myostatin, so that CLI itself and all treatments increase the myostatin/follistatin ratio, thus presumably counteracting late myofiber repair The muscle tissues were subjected to immunohistochemistry for follistatin. ND-UT: non-diabetic, non-SFAL, untreated; UT: SFAL, untreated; SC: SFAL treated with MDSC alone; SC+Mol: SFAL treated with MDSC and molsidomine; Mol: SFAL treated with molsidomine alone. **: p<0.01

FIG. 17 T2DM increases fibrosis and reduces Oct 4+ endogenous SC and low dose pioglitazone prevents these effects. Renal glomerular tissue in a ZDF rat model, treated for 5 months with pioglitazone (PGT). LZR: non-diabetic control. *; p<0.05; **: p<0.01.

### DETAILED DESCRIPTION

### Example 1

Anti-myostatin Strategy for the Therapy of CLI, PAD, or Other Ischemic Conditions of the Skeletal Muscle, based on the Finding of an Overexpression of Myostatin and Increase of the Mst/Fst Ratio in the Skeletal Muscle During CLI as a Key Factor in the Interruption of Myogenic Repair and the Development of Interstitial Fibrosis in the Ischemic Muscles

CLI was induced by ULFA in db/db diabetic mice and treated as follows (n=8/group), for studying the effect of a long-acting NO donor, molsidomine: 1) Untreated (UT): vehicle; 2) MDSC (SC): MDSC into gastrocnemius; 3) MDSC-molsidomine (SC+Mol): as #2, and IP molsidomine (5 mg/kg/day); 4) Molsidomine (Mol) as in #3, no MDSC. Non-diabetic non-ULFA untreated (ND-UT) mice were controls.

At 3 weeks after initiating treatment, mortality, leg preservation, and limb loss/motion/ischemia were determined. Stem cell, myogenic, angiogenic, lipofibrosis, and neurogenesis markers were assayed in muscle tissue by quantitative histochemistry and immunohisto-chemistry and western blot. The results obtained were as follows:

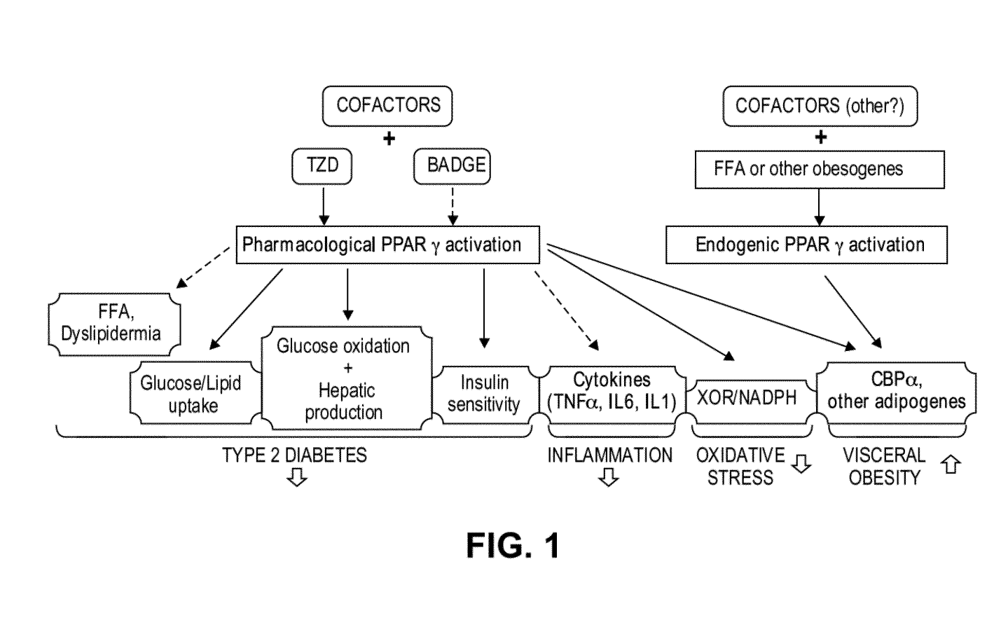

FIG. 1 shows systemic effects of ligand binding to PPARγ in diabetes and obesity.

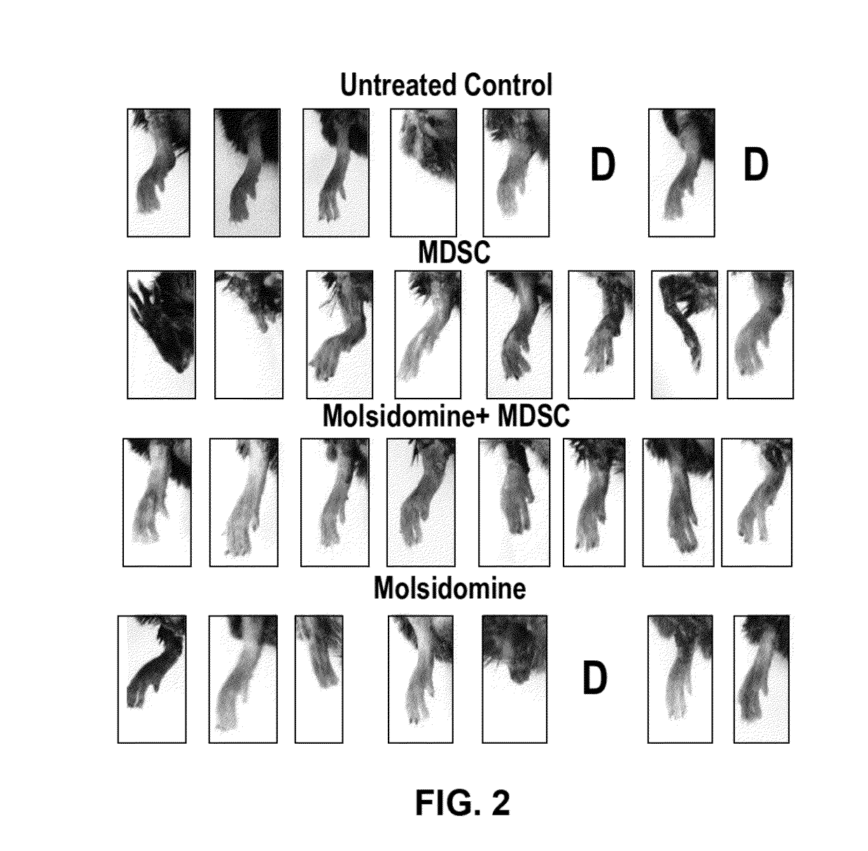

FIG. 2. In comparison to UT, MDSC only slightly reduced mortality and leg loss. MDSC was not very effective in preserving plantar motion or decreasing visual ischemia in the leg denoted by decoloration (not shown).

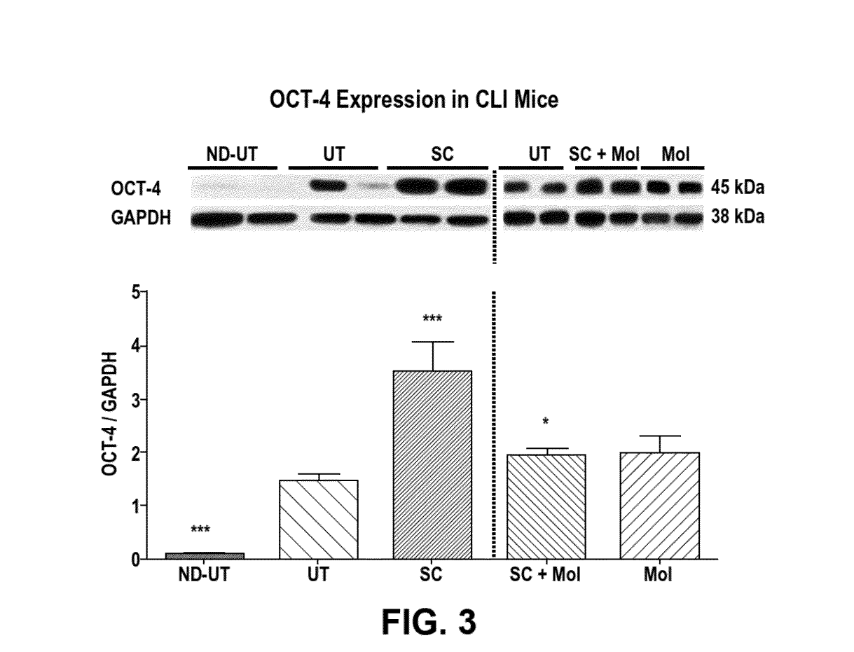

FIG. 3. As expected, MDSC considerably stimulated stem cell number as evidenced by the expression of the key stem cell marker nuclear Oct 4a in the skeletal muscle shown by quantitative western blot, and in contrast Mol reduced this effect.

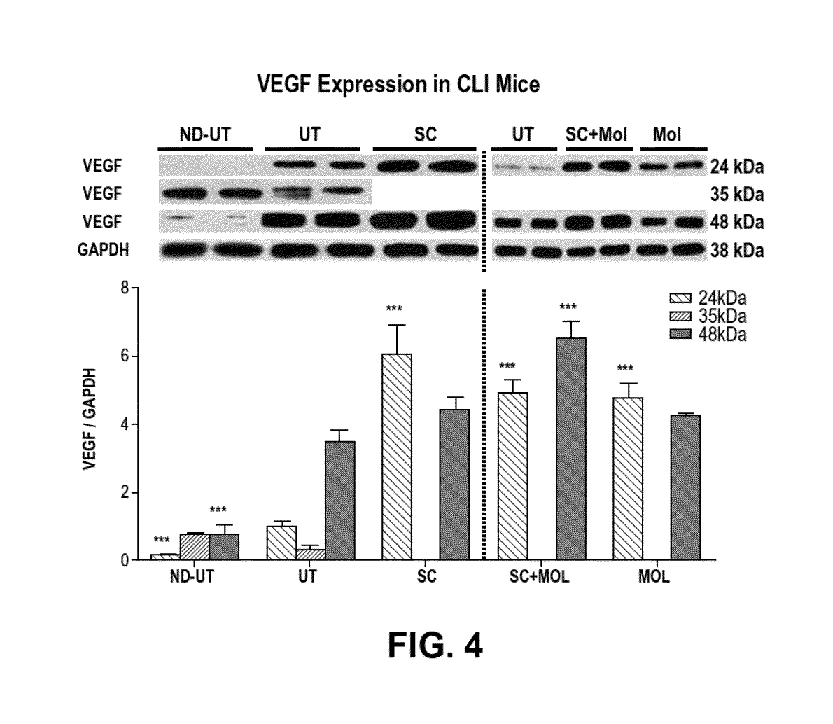

FIG. 4. As expected, MDSC was strongly angiogenic, as shown by the considerable induction of vascular endothelial growth factor (VEGF) expression in the skeletal muscle visualized by quantitative western blot, that was slightly stimulated by the combination with molsidomine. Molsidomine alone was also angiogenic.

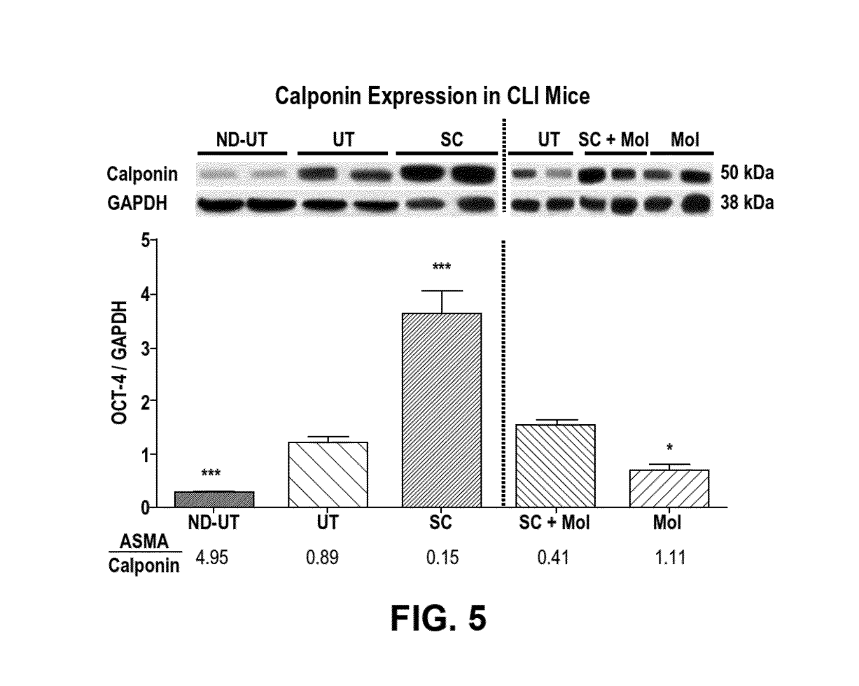

FIG. 5. The pro-angiogenic effects of MDSC were confirmed by the increase in vascular smooth muscle indicated by the specific marker calponin in the skeletal muscle, shown by quantitative western blot, whereas molsidomine abolished the effect.

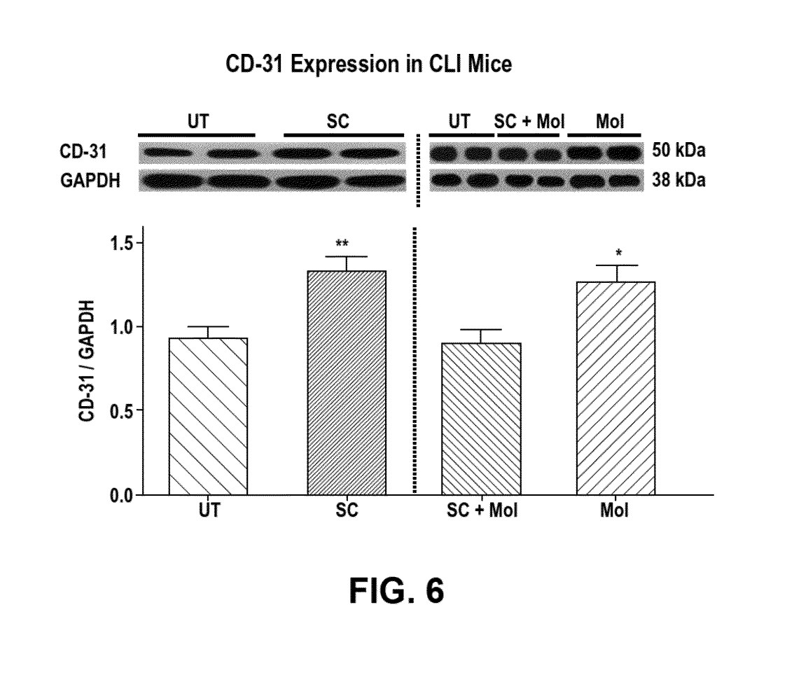

FIG. 6. This was confirmed by the increase in vascular endothelium in the skeletal muscle, shown by quantitative western blot for CD-31 expression, that was elicited by MDSC and also by molsidomine alone.

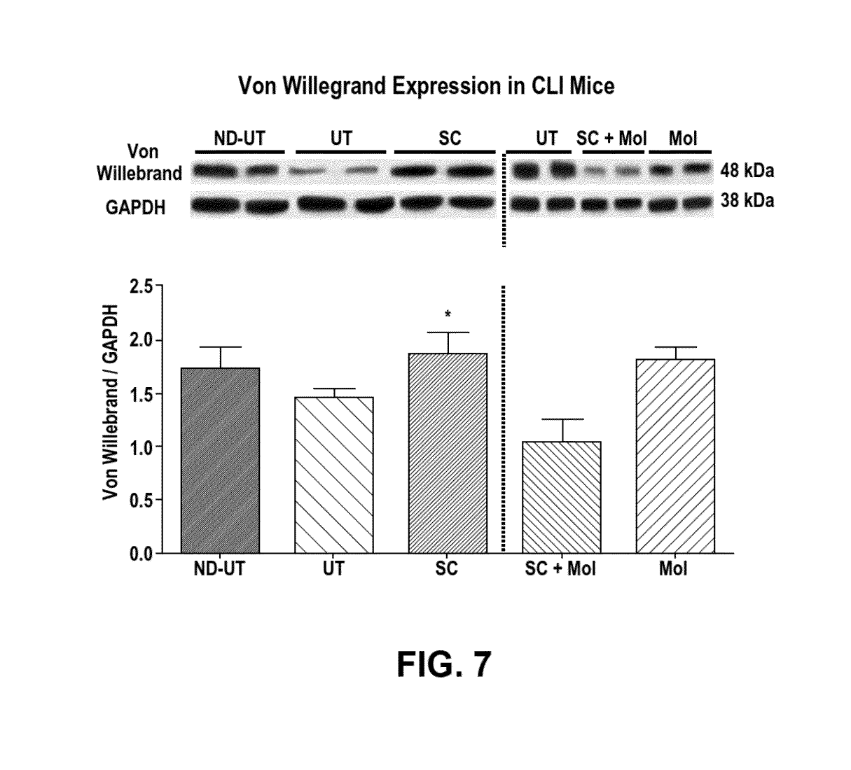

FIG. 7. The pro-angiogenic effects of both MDSC and molsidomine alone were also confirmed by the increase in vascular endothelium in the skeletal muscle, shown by quantitative western blot for von Willebrandt factor expression.

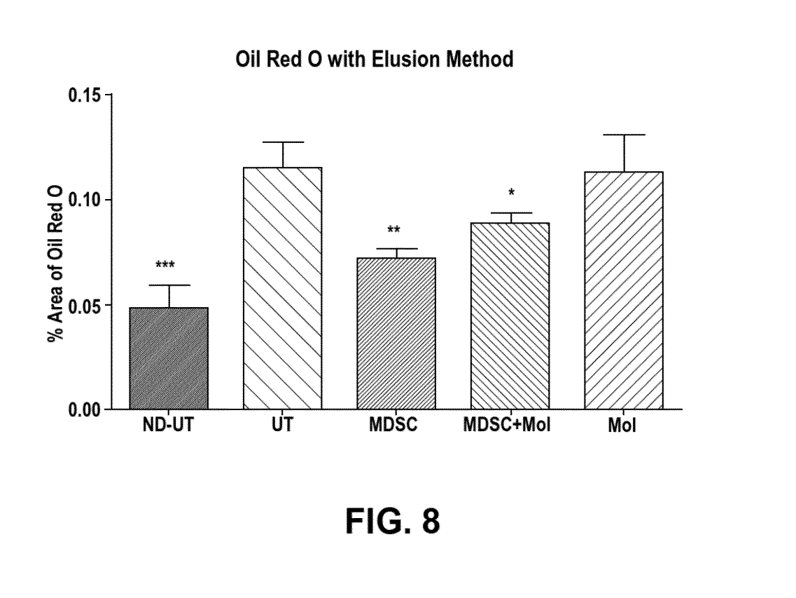

FIG. 8. The protective and repair capacity of MDSC alone, and in the presence of molsidomine, were shown by quantitative immunohistochemistry for Oil Red O that denotes fat infiltration in the skeletal muscle, whereas molsidomine alone did not reduce lipodegeneration of the muscle.

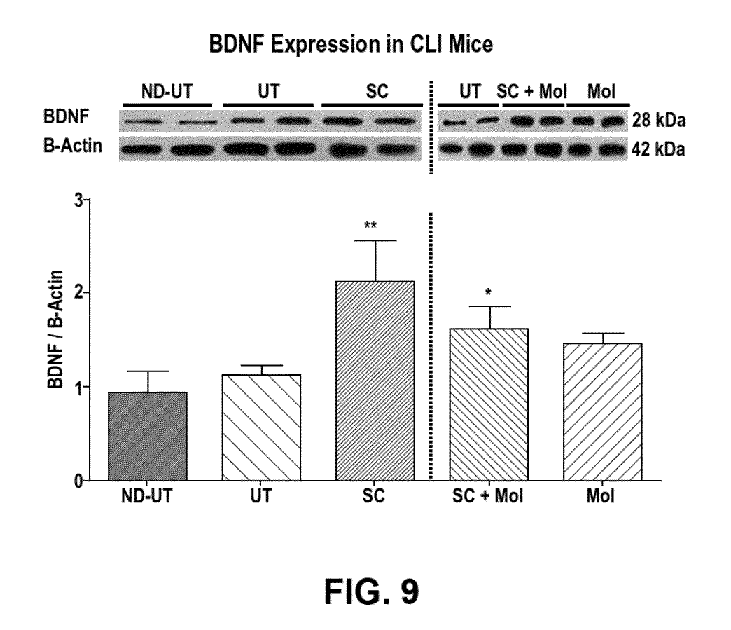

FIG. 9. MDSC also promoted neurotrophic secretion in the skeletal muscle, evidenced by quantitative western blot, specifically brain derived neurotrophic factor (BDNF), that was reduced by combination with molsidomine, but not sufficient to increase nerve terminals (NF70) (not shown).

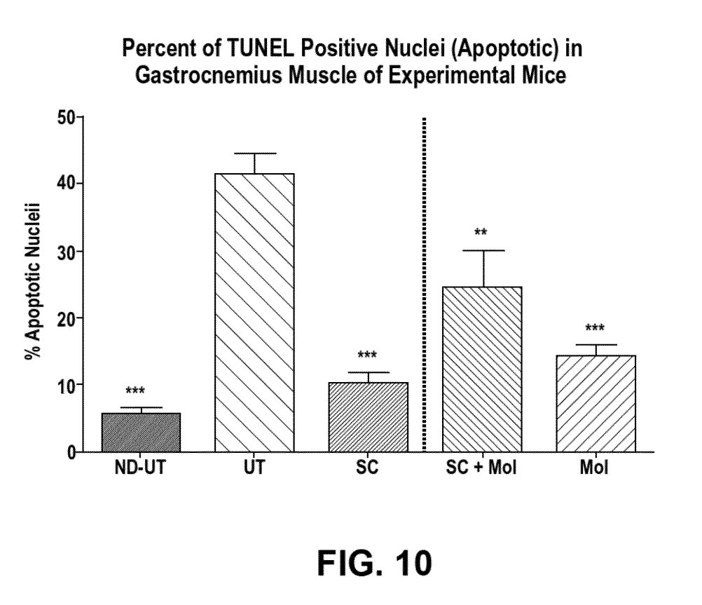

FIG. 10. The previous results showing the expected beneficial effects on the skeletal muscle of MDSC treatment extended to the protection against programmed cell death or apoptosis, evidenced by the reduction in the apoptotic index in the quantitative immunohistochemistry by the TUNEL reaction, that was also exerted by the other treatments but to a lesser degree.

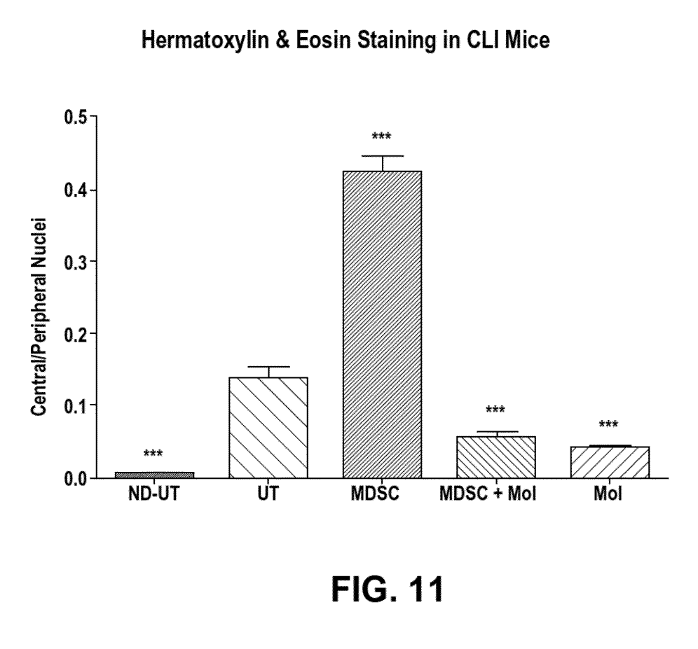

FIG. 11. MDSC caused a dramatic increase in the number of central nuclei a marker of the early stage of myofiber repair, namely the fusion of satellite cell nuclei, that was unexpectedly blocked by combination with molsidomine. Molsidomine alone was even slightly inhibitory.

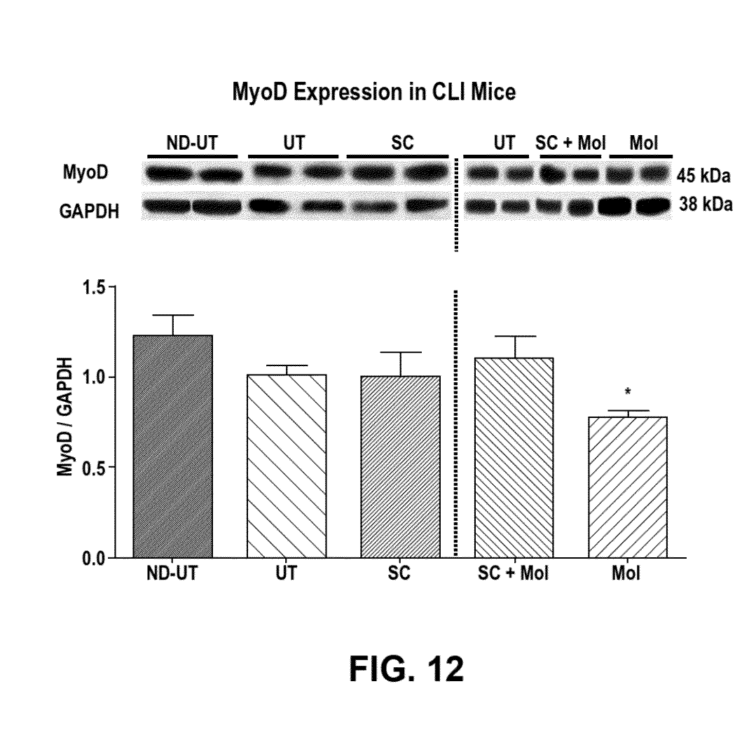

FIG. 12. Unexpectedly, no stimulation was induced by MDSC on the expression in the skeletal muscle of an early myogenic gene, MyoD, as evidenced by quantitative western blot, and the same occurred with the other treatments, thus denoting an interruption of the early myofiber repair denoted by central nuclei.

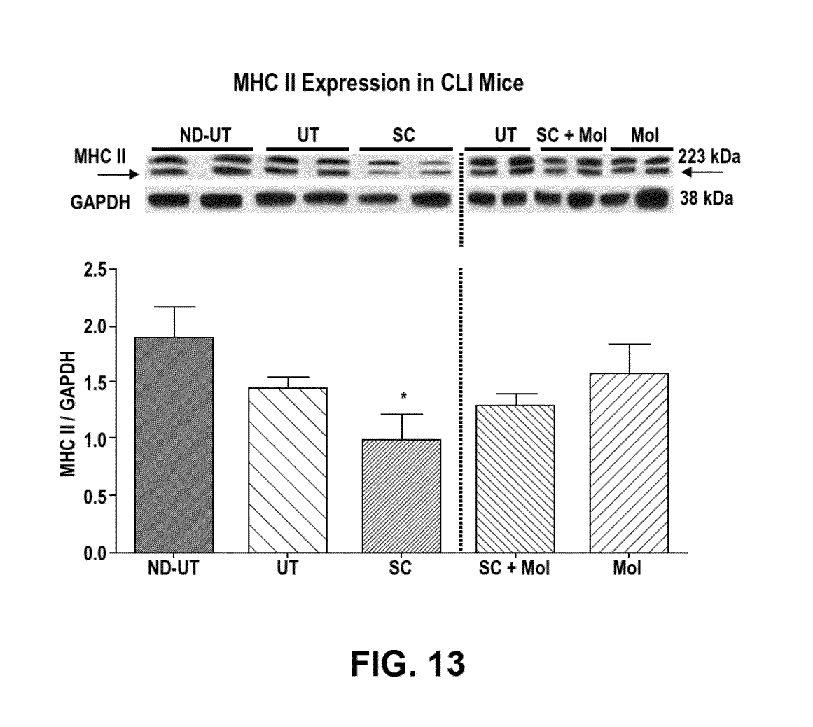

FIG. 13. This failure of all treatments to lead to a final myofiber repair was confirmed by the lack of stimulation of the expression of the late myogenic gene, MHC-II, that is a marker of a certain type of myofibers.

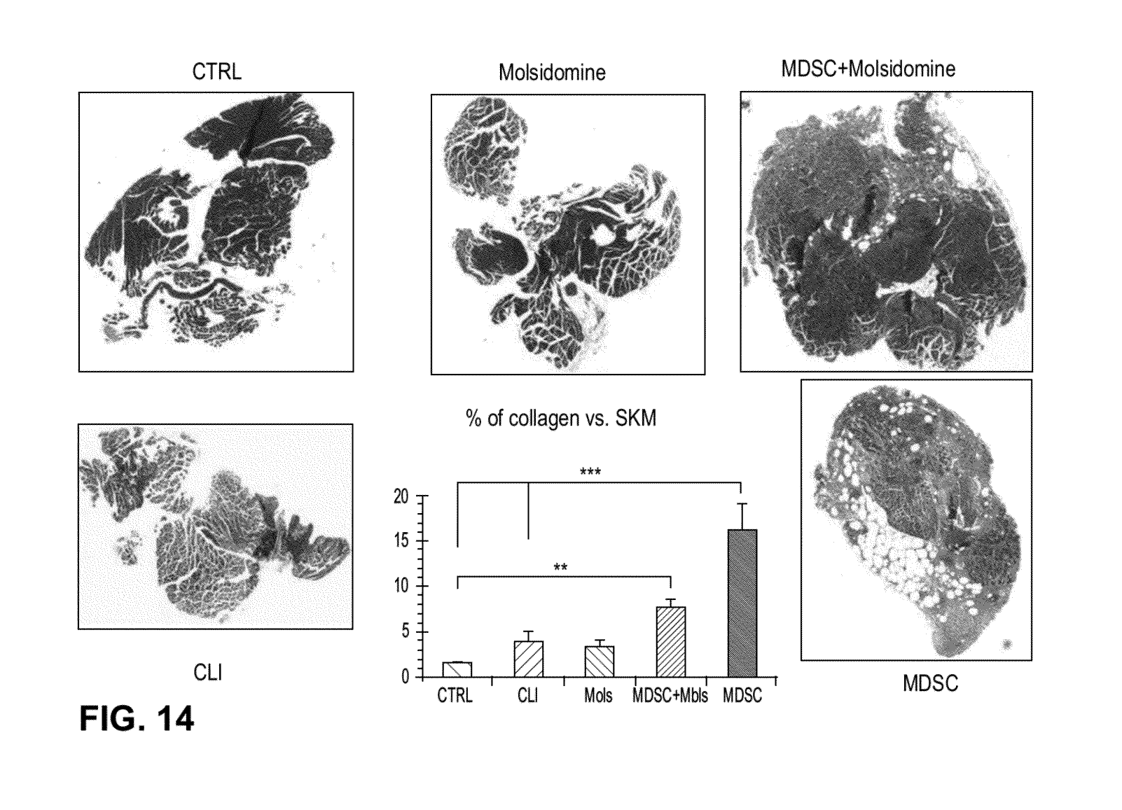

FIG. 14. The interruption of myofiber repair by MDSC and the other treatments was associated with considerable fibrosis in the case of MDSC and MDSC plus molsidomine, although molsidomine alone did not exert this noxious effect, as evidenced by quantitative histochemistry with Masson trichrome staining.

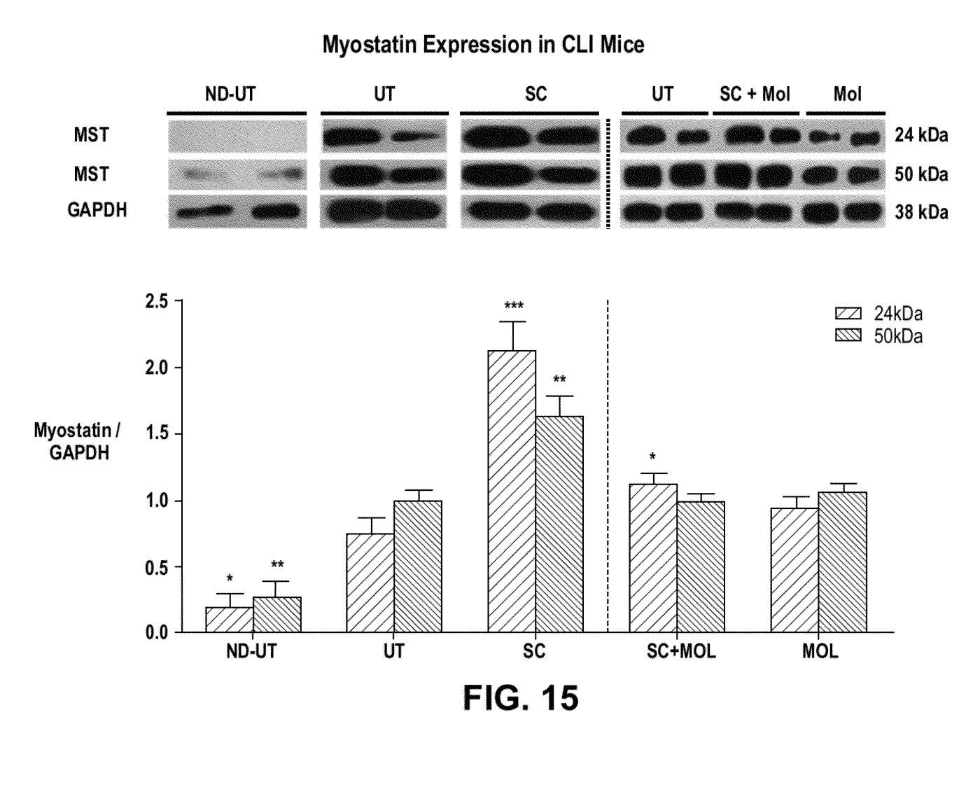

FIG. 15. The counteraction of the mature late myofiber repair by MDSC was accompanied by the increase in the expression in the skeletal muscle of myostatin, that has not been previously reported. Both the 24 and 50 kDa forms expressed in the muscle were measured by quantitative immunohistochemistry. Very significantly, myostatin was increased nearly 4-fold in comparison to the non-diabetic non-UFAL group, a finding that also has not been reported for ischemic conditions affecting the skeletal muscle. Interestingly, the combination with molsidomine reduced the myostatin overexpression, but did not normalize it.

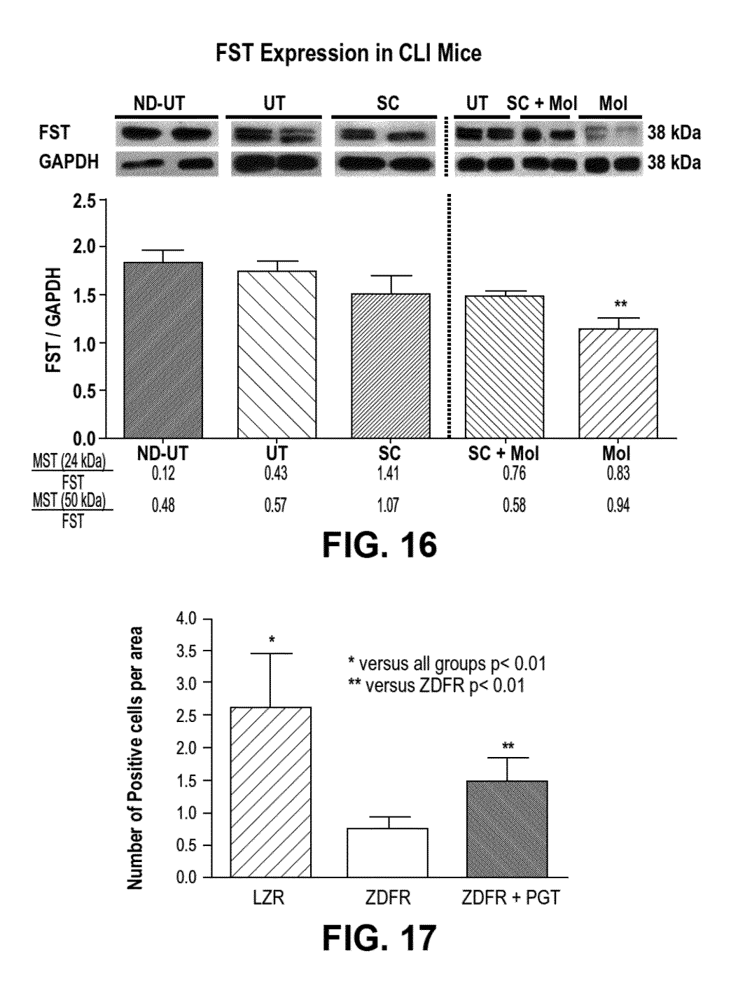

FIG. 16. The best indicator of myostatin activity as an antimyogenic and profibrotic factor in the skeletal muscle is the evaluation of the expression of follistatin, a protein that binds and inactivates myostatin. Although only molsidomine did decrease follistatin, the balance between the muscle inhibitor and its counteractor is indicated by the myostatin/follistatin (Mst/Fst) ratio that remained considerably increased in all treatments as compared to the untreated group. Moreover, there was a marked increase of this ratio in the untreated CLI group as compared to the non-diabetic non-UFAL group.

FIG. 17 shows T2DM increases fibrosis and reduces Oct 4+ endogenous SC and low dose pioglitazone prevents these effects.

In conclusion, the results presented above from a mid-stage (3 weeks) process of skeletal muscle repair in critical limb ischemia in the UFAL diabetic mouse model indicate that MDSC implanted in the muscle are effective in reducing myofiber cell death, and stimulating the early myofiber repair, angiogenesis and neurogenesis, while inhibiting fat infiltration, and this may contribute to leg and motion preservation and reduction in mortality. However, the failure of MDSC, or of stem cells in general, to complete the early repair by increasing myogenesis and mature myofiber replacement, and the associated induction of excessive collagen deposition is associated with the MDSC triggering over-expression of the main inhibitor of muscle mass and a key profibrotic effector, myostatin, without the corresponding follistatin compensation. Unexpectedly, the supplementation of MDSC with a long-acting nitric oxide donor failed to stimulate their beneficial effects, and other than supporting angiogenesis and inhibiting cell death does not seem at this stage to be justified, although a late stage of regeneration (8 weeks) may reveal beneficial effects for the combination. There is no similar study with anti-myostatin strategy on skeletal muscle necrosis and fibrosis in CLI. PAD, or other ischemic conditions.

Therefore, the pharmacological stimulation of MDSC for the therapy of CLI, and in general of other stem cells, should be based on combating myostatin over-expression. Specifically, the following modalities of treatment are proposed to improve myofiber formation and reduce fibrosis in the skeletal muscle: a) follistatin; b) decorin; c) myostatin antibodies; d) myostain propeptide; e) ligands of the myostatin receptor, the activin type IIb receptor (ActIIbR); f) antisense or shanty-myostatin; g) combination of the anti-myostatin strategy with the use of stem cells.

### Example 2

Continuous Long-term Administration of PPARγ Agonists at Low Doses not Affecting Glycemic Control, Either Alone or in Combination with Stem Cells, for the Treatment of the Necrotic Skeletal Muscle in CLI, PAD, or any Other Ischemic Condition, Based on the Antifibrotic, and Anti-inflammatory Effects, and Stem Cell Modulation of PPARγ Agonists.

Pioglitazone is a potential adjuvant, since in other systems it is shown that a low oral dose (0.6 mg/kg/day, equivalent to about 6 mg in humans) that does not exert glycemic control or increase body weight, prevents functional disorders and their underlying histopathology in the kidney and corpora cavernosa in rat models for insulin resistance and T2DM, by ameliorating chronic inflammation, lipofibrosis, and oxidative stress. This low dose leaves glycemia at around 250-300 mg/dl, but eliminates the risk of side effects observed with the glycemia-normalizing dose of 12 mg/kg/day, such as obesity and fat infiltration in the tissues.

This low dose reduced pro-fibrotic connective tissue growth factor expression in the kidney in diabetic nephropathy and a number of other anti-inflammatory and antioxidant markers. We have demonstrated the beneficial direct effect of pioglitazone on stem cell content by the counteraction of the decrease of Oct 4+ nuclei in the kidney caused by T2DM (FIG. 17). Expression of nuclear Oct 4, and its corresponding 45 kDa isoform Oct 4A in western blots, as opposed to the cytoplasmic Oct 4B, is a marker of endogenous stem cells, was also shown, and this therefore confirms the effective protection of stem cells survival and “stemness” by PPARγ agonists.

Specifically, the following modalities of treatment are proposed:

a) oral pioglitazone at 10 mg/kg/day or lower doses not exerting glycemic control, daily, for at least 2 months;

b) oral pioglitazone at doses higher than 10 mg/kg/day exerting glycemic control, daily, for at least 2 months;

c) as a) or b), but combined with stem cells given to the skeletal muscle.

A reason why stem cell and other therapies are ineffective or have very limited efficacy not just for CLI, but for other milder forms of peripheral artery disease (PAD) and general ischemic conditions affecting the skeletal muscle is that the current therapeutic approaches aim virtually exclusively to stimulate angiogenesis, or blood vessel formation. This is insufficient to repair the target tissue, namely the skeletal muscle that suffers the necrosis that leads to the loss of limb function and eventually the legs and feet themselves, and that therapies must aim to repair simultaneously multiple tissues in the limbs but focus predominantly on skeletal muscle regeneration and not just on the blood vessels. This involves the concerted repair of the damaged myofibers and the inhibition of the lipofibrotic degeneration, or the deposit of excessive collagen and fat in the interstitial tissue and in the myofibers, that is associated with the inadequate healing occurring in the bouts of spontaneous or pharmacologically induced tissue regeneration.

The examples presented above show for the first time in the ischemic disease literature that CLI, as studied in a diabetic mouse model model where the damage is induced in one leg by unilateral femoral artery ligation (UFAL), that ischemia induces in the affected skeletal muscle a substantial overexpression of the main inhibitor of skeletal muscle mass, namely, myostatin, and that this is not compensated by the overexpression of its main antagonist, follistatin, so that there is a significant increase in the myostatin/follistatin (Mst/Fst) ratio. Moreover, also for the first time, it is shown that stem cells, in this case muscle derived stem cells (MDSC) implanted therapeutically in the gastrocnemius muscle lead to an even more considerable induction of myostatin and the increase of the Mst/Fst ratio. This is associated with the expected beneficial stimulation of stem cell number (implanted and endogenous), angiogenesis, neurogenesis, and the initial phase of tissue repair evidenced by the increase in central nuclei in the myofibers and the inhibition of programmed cell death (apoptosis) and fat infiltration, but also with a counterproductive or noxious interruption of myofiber formation at an early stage as evidenced by the lack of increase of early and late myogenesis, as well as another deleterious increase in tissue fibrosis.

The results additionally indicate that an initially promising therapeutic approach for skeletal muscle repair in CLI, the use of a long-acting nitric oxide donor (molsidomine), either alone or in combination with MD SC, that was expected to stimulate satellite cell nuclei fusion into myofibers and the MDSC myogenic, angiogenic and neurogenic differentiation, and inhibit lipofibrosis, fails to exert any beneficial effects over MDSC alone, and/or to combat the noxious increase in the Mst/Fst.

A certain class of drugs named thiazolidinediones, which are ligands of the PPARγ, and are used for the treatment of T2DM, specifically pioglitazone or Actos, are antifibrotic, anti-inflammatory, and antioxidative stress, in the penile corpora smooth muscle in T2DM and aging and in the kidney in diabetic nephropathy, and stimulate endogenous stem cells number in these tissues, when given long-term continuous at low doses that do not exert glycemic control or, as a side effect, promote fat accumulation and obesity. Similar effects may occur in the skeletal muscle affected by ischemia, where a similar strategy is anticipated to result in a beneficial antifibrotic, anti-inflammatory, and antioxidative stress paradigm stimulating the repair of the skeletal muscle.

In view of the above description, a method for the treatment or prevention of critical limb ischemia (CLI), peripheral arterial disease (PAD) and any other ischemic condition affecting the skeletal muscle preferentially in limbs but in general throughout the body, that replaces the current medical approaches focusing primarily and often exclusively on pro-angiogenic interventions to induce neo-angiogenesis or neo-arteriogenesis, is described that focuses primarily on the repair and regeneration of the damaged myofibers in the skeletal muscle and the counteraction of chronic inflammation and fibrosis within a targeted pro-myogenesis approach that simultaneously promotes a multiple process of angiogenesis, neurogenesis, and regeneration of other tissues in the affected skeletal muscle.

According to one embodiment, the method includes the long-term local or systemic continuous administration (e.g., months, years according to a regimen, schedule or period) of agents that reduce or inhibit the activity or the protein expression of myostatin, also named growth differentiation factor 8 (GDF-8), to prevent or combat the overexpression of myostatin in the ischemic muscle, in conjunction with the concurrent local or systemic single or multiple administration of stem cells of any nature, including but not limited to adult or embryonic stem cells, or induced pluripotent stem cells (iPS). Such local or systemic anti-myostatin approaches include, but are not limited to: a) follistatin, b) decorin; c) myostatin antibodies; d) myostain propeptide; e) myostatin shRNA or antisense RNA; f) myostatin microRNAs inhibiting myostatin expression or anti-microRNAs stimulating this expression; g) ligands of the myostatin receptor, the activin type IIb receptor (ActIIbR).

According to another embodiment, the method includes the long-term local or systemic continuous administration (e.g., months, years according to a regimen, schedule or period) of a thiazolidenedione or other PPAR gamma agonist at low doses that do not exert glycemic control or induce overweight, in an approach not focused on treating insulin resistance or type 2 diabetes but aimed on ischemic skeletal muscle regeneration, to combat the chronic inflammation and fibrosis in ischemic skeletal muscle, and induce stem cell trophic activity on stem cells promoting myofiber repair, in conjunction with the concurrent local or systemic single or multiple administration of stem cells of any nature, including but not limited to adult or embryonic stem cells, or induced pluripotent stem cells (iPS). Representatively, the doses of PPAR gamma agonists are such that they induce glycemic control and/or may cause overweight, but the approach is also not focused on treating insulin resistance or type 2 diabetes, but ischemic skeletal muscle regeneration.

According to the embodiments, the agents described in such methods can be used in combination, namely anti-myostatin and PPAR gamma agonist given together with stem cells. Still further, the agents can be used either alone or in combination but without concurrent stem cell administration.

Still further embodiments include a kit or kits for use in treating or inhibiting an ischemic condition affecting the skeletal muscle. In one embodiment, a kit includes an agent having a property to inhibit an activity or protein expression of myostatin or growth differentiation factor 8 (GDF-8) and instructions for administration of a dosage of that quantity according to a long term continuous regimen. Representative agents include a) follistatin, b) decorin; c) myostatin antibodies; d) myostain propeptide; e) myostatin shRNA or antisense RNA; f) myostatin microRNAs inhibiting myostatin expression or anti-microRNAs stimulating this expression; or g) ligands of the myostatin receptor, the activin type IIb receptor (ActIIbR). In another embodiment, a kit for use in treating or inhibiting an ischemic condition affecting the skeletal muscle includes a quantity of a thiazolidenedione or other PPAR gamma agonist at a dosage that does not exert glycemic control or induce overweight and instructions for administration of a dosage of that quantity according to a long term continuous regimen. In yet another embodiment, a kit includes a quantity of an agent having a property to inhibit an activity or a protein expression of myostatin or growth differentiation factor 8 (GDF-8) and a quantity of a thiazolidenedione or other PPAR gamma agonist at a dosage that does not exert glycemic control or induce overweight and instructions for administration of a dosage of each quantity according to a long term continuous regimen.

In the description above, for the purposes of explanation, numerous specific details have been set forth in order to provide a thorough understanding of the embodiments. It will be apparent however, to one skilled in the art, that one or more other embodiments may be practiced without some of these specific details. The particular embodiments described are not provided to limit the invention but to illustrate it. The scope of the invention is not to be determined by the specific examples provided above but only by the claims below. In other instances, well-known structures, devices, and operations have been shown in block diagram form or without detail in order to avoid obscuring the understanding of the description. Where considered appropriate, reference numerals or terminal portions of reference numerals have been repeated among the figures to indicate corresponding or analogous elements, which may optionally have similar characteristics.

It should also be appreciated that reference throughout this specification to “one embodiment”, “an embodiment”, “one or more embodiments”, or “different embodiments”, for example, means that a particular feature may be included in the practice of the invention. Similarly, it should be appreciated that in the description various features are sometimes grouped together in a single embodiment, figure, or description thereof for the purpose of streamlining the disclosure and aiding in the understanding of various inventive aspects. This method of disclosure, however, is not to be interpreted as reflecting an intention that the invention requires more features than are expressly recited in each claim. Rather, as the following claims reflect, inventive aspects may lie in less than all features of a single disclosed embodiment. Thus, the claims following the Detailed Description are hereby expressly incorporated into this Detailed Description, with each claim standing on its own as a separate embodiment of the invention.

1. A method for treating or inhibiting an ischemic condition affecting the skeletal muscle comprising administering an agent having a property to inhibit an activity or a protein expression of myostatin or growth differentiation factor 8 (GDF-8) according to a regimen to treat or inhibit the ischemic condition.

2. The method of claim 1, wherein administering comprises administering in conjunction with the concurrent local or systemic single or multiple administration of stem cells.

3. The method of claim 1, wherein the regimen is a long term continuous regimen.

4. The method of claim 1, wherein the agent comprises a) follistatin, b) decorin; c) myostatin antibodies; d) myostain propeptide; e) myostatin shRNA or antisense RNA; f) myostatin microRNAs inhibiting myostatin expression or anti-microRNAs stimulating this expression; or g) ligands of the myostatin receptor, the activin type IIb receptor (ActIIbR).

5. The method of claim 1, further comprising administering a thiazolidenedione or other PPAR gamma agonist at a dosage that does not exert glycemic control or induce overweight.

6. The method of claim 5, wherein administering comprises administering in conjunction with the concurrent local or systemic single or multiple administration of stem cells and the dosage of the thiazolidenedione or other PPAR gamma agonist induce stem cell trophic activity on stem cells promoting myofiber repair.

7. A method for treating or inhibiting an ischemic condition affecting the skeletal muscle comprising administering an effective amount of a thiazolidenedione or other PPAR gamma agonist at a dosage that do not exert glycemic control or induce overweight.

8. The method of claim 7, wherein administering comprises administering in conjunction with the concurrent local or systemic single or multiple administration of stem cells and the dosage of the thiazolidenedione or other PPAR gamma agonist induces stem cell trophic activity on stem cells promoting myofiber repair.

9. The method of claim 7, wherein administering comprises administering according to a long term continuous regimen.

10. The method of claim 7, further comprising administering an agent having a property to inhibit an activity or a protein expression of myostatin or growth differentiation factor 8 (GDF-8).

11. A kit for use in treating or inhibiting an ischemic condition affecting the skeletal muscle comprising a quantity of an agent having a property to inhibit an activity or a protein expression of myostatin or growth differentiation factor 8 (GDF-8) and instructions for administration of a dosage of that quantity according to a long term continuous regimen.

12. The kit of claim 11, wherein the agent comprises a) follistatin, b) decorin; c) myostatin antibodies; d) myostain propeptide; e) myostatin shRNA or antisense RNA; f) myostatin microRNAs inhibiting myostatin expression or anti-microRNAs stimulating this expression; or g) ligands of the myostatin receptor, the activin type IIb receptor (ActIIbR).

13. The kit of claim 11, further comprising a quantity of a thiazolidenedione or other PPAR gamma agonist at a dosage that does not exert glycemic control or induce overweight.

14. A kit for use in treating or inhibiting an ischemic condition affecting the skeletal muscle comprising a quantity of a thiazolidenedione or other PPAR gamma agonist at a dosage that does not exert glycemic control or induce overweight and instructions for administration of a dosage of that quantity according to a long term continuous regimen.

15. The kit of claim 14, further comprising a quantity of an agent having a property to inhibit an activity or a protein expression of myostatin or growth differentiation factor 8 (GDF-8) and instructions for administration of a dosage of that quantity according to a long term continuous regimen.

16. The kit of claim 14, wherein the agent comprises a) follistatin, b) decorin; c) myostatin antibodies; d) myostain propeptide; e) myostatin shRNA or antisense RNA; f) myostatin microRNAs inhibiting myostatin expression or anti-microRNAs stimulating this expression; or g) ligands of the myostatin receptor, the activin type IIb receptor (ActIIbR).
